(19) United States
(12) Patent Application Publication
(10) Pub. No.: US 2025/0255856 A1
(43) Pub. Date: Aug. 14, 2025

(54) THERAPEUTIC COMPOSITIONS WITH IMINO SUGARS FOR THE TREATMENT OF DISEASES WITH ACCUMULATION OF HEPARAN SULFATE

(71) Applicants: Luigi Michele Pavone, Napoli (IT); Annalisa Guaragna, Napoli (IT); Valeria De Pasquale, Napoli (IT); Anna Esposito, Napoli (IT); Massimo D'Agostino, Santa Maria Capua Vetere (CE) (IT)

(72) Inventors: Luigi Michele Pavone, Napoli (IT); Annalisa Guaragna, Napoli (IT); Valeria De Pasquale, Napoli (IT); Anna Esposito, Napoli (IT); Massimo D'Agostino, Santa Maria Capua Vetere (CE) (IT)

(21) Appl. No.: 18/856,892

(22) PCT Filed: Apr. 18, 2023
(86) PCT No.: PCT/EP2023/059966

§ 371 Date: Oct. 15, 2024

(30) Foreign Application Priority Data
Apr. 20, 2022 (IT) 102022000007808

(51) Int. Cl. A61K 31/445; A61P 35/00; C07D 211/46

(52) U.S. Cl. CPC A61K 31/445; A61P 35/00; C07D 211/46

(57) ABSTRACT

Compositions herein disclosed are conceived for the treatment and prevention of diseases caused by accumulation of heparan sulfate including mucopolysaccharidosis, Alzheimer's disease and cancers. These compositions include as active ingredient an iminosugar belonging to L-steric series and derivatives thereof. The L-iminosugars of this invention are able to reduce the levels of heparan sulfate in cells of patients affected by mucopolysaccharidosis and cancer, and to reduce the accumulation of amyloid plaques in a model of neurodegenerative disease. Therefore, the use of these compounds prevents the onset of symptoms associated with these diseases, thus improving the quality and length of life of patients suffering from diseases characterized by accumulation of heparan sulfate.

The present invention relates to specific iminosugars belonging to the L-steric series and their pharmaceutically acceptable salts for use in the treatment and prevention of diseases caused by accumulation of heparan sulfate, in particular mucopolysaccharidosis, Alzheimer's disease and cancer.

### BACKGROUND OF THE INVENTION

Mucopolysaccharidoses (MPS) are hereditary metabolic diseases caused by the absence or deficiency of lysosomal enzymes necessary for the catabolism of glycosaminoglycans (GAGs), heparan sulfate (HS), dermatan sulfate (DS), keratan sulfate (KS), chondroitin sulfate (CS) and hyaluronic acid (HA) [Neufeld, E. F. and Muenzer, E. F. The mucopolysaccharidoses, in: Scriver, C. R. et al (Eds), The Metabolic and Molecular Bases of Inherited Diseases, McGraw-Hill, 2001, pp. 3421-3452]. The lack of these lysosomal enzymes causes accumulation of non-degraded GAGs in various cellular compartments and multiple organ and system dysfunctions, with distinct clinical manifestations depending on the type of the defective enzyme and accumulated GAG. MPS are therefore classified in eleven different diseases (MPS I, II, IIIA, IIIB, IIIC, IIID, IVA, IVB, VI, VII and IX) depending on the defective lysosomal enzyme and in seven subtypes if we consider the accumulated products: MPS I-heparan and dermatan sulfate; MPS II-heparan and dermatan sulfate; MPS III-heparan sulfate; MPS IV-keratan sulfate and chondroitin 6-sulfate; MPS VI-dermatan sulfate; MPS VII-heparan sulfate, dermatan sulfate and chondroitin 6-sulfate; MPS IX-hyaluronic acid. GAG accumulation on cell membrane and within lysosomes, together with other pathogenetic mechanisms, lead to various clinical consequences, with a wide phenotypic variability [Mehta, A. and Winchester, B. Lysosomal Storage Disorders: A Practical Guide, First Edition 2012]. Typical clinical symptoms of the disease include neurological disorders, cardiovascular dysfunction, skeletal, joint, airway, hearing and vision defects, and death in the second or third decade of life [Oussoren E. et al. (2011). Biochim. Biophys. Acta 1812, 1542; Schiattarella G. G. et al. (2015). PLOS One 10, e0131662; Costa, R. et al. (2017). Hum. Mol. Genet. 26, 1643; Bellettato, C. M. and Scarpa, M. J. (2010). Inherit. Metab. Dis. 33, 347].

Timely diagnosis is essential for MPS patients and only few therapeutic strategies are currently available with variable and limited efficacy [Hollak C E M, Wijburg F A. (2014) J Inherit metab dis. 37, 587]. Current therapeutic options for MPS include enzyme replacement therapy (ERT), substrate reduction therapy (SRT), pharmacological chaperone therapy (PCT), gene therapy (GT), and hematopoietic stem cell transplantation (HSCT). [Fecarotta, S. et al. (2018). Ital. J. Pediatr. 44, 124; Poswar, F. et al. (2017). Expert Opin. Investig. Drugs 26, 1331]. Most of these strategies are described in US2013302308, WO2012177778, U.S. Pat. No. 8,623,910, US2012190642, US2011008810, EP2081023, KR100762945, KR20040084881, U.S. Pat. No. 8,105,788, US2009092996, WO02055064, RU2196988, RU2083205, JP2008102114, JP4965999, JP2003265196. These therapeutic treatments have several limitations. Particularly, ERT, which is the most used, is unable to correct all the defects associated with these pathologies, especially those related to the central nervous system due to the inability of the recombinant enzymes to overcome the blood-brain barrier. Treatment with stem cells is also ineffective, but above all it is extremely dangerous due to the uncertain fate of stem cells after administration to the patient, including the possibility that these cells acquire a tumor phenotype. GT is still not in use in the clinic today due to the high immunogenicity of the vectors and the dangers related to the integration of the viral genome, albeit inactive, into the genome of treated patients. Due to the limitations of such strategies, scientific research continues to study MPS pathophysiology for the identification of new therapeutic strategies.

Based on these considerations, it can be deduced that MPS do not currently find completely adequate therapies to reduce or eliminate the serious symptoms of patients affected by these diseases.

The potential use of GAG biosynthesis inhibitors has been also tested in MPS diseases [Fecarotta, S. et al. (2018). Ital. J. Pediatr. 44, 124; Poswar, F. et al. (2017). Expert Opin. Investig. Drugs 26, 1331]. This approach, known as “substrate-reduction therapy” (SRT), employs small molecules able to cross the blood-brain barrier, thus having the potential to treat the neurological phenotype of the disease. The first molecule identified as a potential drug for SRT in MPS patients with neurological manifestations was genistein, a soy-derived isoflavone with structural similarity to 17β-estradiol, which inhibits GAG synthesis by affecting the epidermal growth factor (EGF)-dependent molecular signaling pathway [Jakobkiewicz-Banecka, J. et al. (2009). J. Biomed. Sci. 16, 26]. However, genistein has been shown to be ineffective in clinical trials in patients with MPS III. The identification of novel molecules that interfere with GAG synthesis may provide a useful tool to improve the neurological phenotype in MPS patients. On the other hand, manipulation of GAG synthesis in several diseases has been performed using synthetic xylosides that reduce GAG bound to proteoglycans, especially HS, thus modulating the biological functions of HSPGs (HS proteoglycans) [Chua, J. S. and Kuberan, B. (2017). Acc. Chem. Res. 50, 2693].

Due to the ability of HSPGs to regulate multiple cellular functions including cell proliferation, differentiation, adhesion, migration, survival, and signaling, these complex molecules have emerged as potential therapeutic targets for the treatment of several diseases, including cancer, inflammation, infection, wound closure, lung disease, Alzheimer's disease and other diseases [Varki, A. et al., Essentials of Glycobiology, 2nd ed., Cold Spring Harbor Laboratory Press, New York, 2009].

In the last decades, HSPGs have been an intriguing object of study due to their complex structural features, their finely regulated biosynthetic mechanism, and the wide range of functions they perform in living organisms from development to adulthood. From these studies, key roles of HSPGs in cancer initiation and progression emerged and are currently being explored as potential biomarkers and therapeutic targets for cancers. The multifaceted nature of the structure/activity of HSPGs results in their ability to act as inhibitors or promoters of tumor growth and invasion depending on the tumor type. Dysregulation of the structural and functional characteristics of HSPGs resulting in malignancy may be due both to altered expression levels and to changes in their structure and function as a result of altered activity of their biosynthetic enzymes or modifiers. Indeed, in the tumor microenvironment, HSPGs undergo structural alterations through the displacement of the proteoglycan ectodomain from the cell surface or the fragmentation and/or desulfation of the HS chains, influencing the function of the HSPGs with a significant impact on the molecular interactions between tumor cells and their microenvironment, and the behavior of tumor cells themselves.

Among their functions, HSPGs help many viruses invade host cells at various stages of their life cycle. Viruses use HSPGs for host cell attachment, internalization, intracellular trafficking, egress, and dissemination. Recently, the involvement of HSPGs in the pathogenesis of SARS-CoV-2 infection has been established [De Pasquale, V. et al. (2021). Int J Mol Sci. 22, 6574].

The common link among all the different causes of neuropathology in the Alzheimer's disease brain is the early accumulation of HSPGs and HS glycosaminoglycans. All these events further implicate HSPG/HSGAG as key players in the pathogenesis of neuropathology in Alzheimer's disease.

In recent years, iminosugars have shown considerable pharmacological potential in the management of lysosomal storage disorders (LSD) as result of their ability to interact with carbohydrate-processing enzymes [Nash, R. J. et al. (2011). Future Med. Chem. 3, 1513; Platt, F. M. et al. (2018). Nat. Rev. Dis. Prim. 4, 27; Compain, P.; Martin, O. (2007). “Iminosugars: From Synthesis to Therapeutic Applications” (John Wiley & Sons, Ltd); Butters, T. D. et al. (2003), Curr. Top. Med. Chem. 3, 561]. These glycomimetics have found application in the treatment of LSDs both by inhibiting the accumulation of substrates in lysosomes (SRT) [Platt, F. M. and Jeyakumar, M. (2008). Acta Paediatr. 97, 88; Coutinho, M. F. et al. (2016), Int. J. Mol. Sci. 17, 1065], and for their ability to reversibly bind lysosomal glycosidases at sub-inhibitory concentrations, improving the function of mutant enzymes (PCT) [Sánchez-Fernández, E. M., et al. (2016) Chem. Commun. 52, 5497; Cox, T. M. et al., (2008) “Medicinal use of Iminosugars” in “Iminosugars: From Synthesis to Therapeutic Applications” (John Wiley & Sons, Ltd), pp. 295-326].

Two iminosugars have reached the market for LSD therapy, Miglustat, also known as D-NBDNJ, licensed for the treatment of type I Gaucher disease [Cox, T. M. et al. (2000). The Lancet 355, 1481] and Niemann-Pick type C disease (as SRT therapy) [Pineda, M. et al. (2018). Orphanet J. Rare Dis. 13, 140] and Migalastat, also known as DGJ, the only pharmacological chaperone currently approved and used in Fabry disease [Benjamin, E. R. et al. (2009). J. Inherit. Metab. Dis. 32, 424; Markham, A. (2016) Drugs 76, 1147].

In addition to these iminosugars, a variety of other derivatives have been evaluated for their use as drug candidates in several LSDs, including Pompe disease and MPS [Parenti, G. et al. (2021). EMBO Mol. Med. 13, e12836; Díaz, J. C. L. et al. (2020), Int. J. Mol. Sci. 21, 1]. In this context, a promising activity has been observed for some iminosugars as pharmacological chaperones for the treatment of MPS II, III and IV [Zhu, S. et al. (2021). Chem.-A Eur. J. 27, 11291; Fantur, K. et al. (2010). Mol. Genet. Metab. 100, 262; Thonhofer, M. et al. (2016). Carbohydr. Res. 429, 71; Takai, T. et al. (2013). Mol. Ther. 21, 526].

Moreover, an interesting application of iminosugars in MPS concerns the hypothesis that the secondary storage of gangliosides could represent a therapeutic target in patients with neurological involvement. On this basis, the iminosugar D-NBDNJ (Miglustat) was evaluated as a substrate-reducing agent for MPS type III due to its ability to interfere with glycosphingolipid metabolism [Fecarotta, S. et al. (2018). Ital. J. Pediatr. 44, 124]. Despite the promising results obtained in preclinical studies [Kaidonis, X. et al. (2016). Mol Gen. Metab. 118, 110], no beneficial effects were observed in MPS III patients treated with D-NBDNJ (Miglustat) [Guffon, N. et al. (2011). J. Pediatrician. 159, 838]. These data clearly suggest that iminosugars are attractive candidates for MPS treatment.

In frame of our studies aimed to explore the role of chirality on the pharmacological activity of iminosugars and other bioactive compounds [Esposito, A. et al. (2020). Chem.—A Eur. J. 26, 2597; Esposito, A. et al. (2020) Mar. Drugs 18, 572; Esposito, A. et al., (2019) RSC Adv. 9, 21519] a very promising potential of L-iminosugars in the treatment of rare diseases has recently been highlighted. Particularly, L-NBDNJ, (the enantiomer of D-NBDNJ, Miglustat) has shown interesting potential as a candidate for the combination therapy of Pompe disease, without working as inhibitor of most glycosidases, unlike its D-enantiomer [D'Alonzo, D. et al. (2017). J. Med Chem. 60, 9462].

Even more interesting results were obtained when L-iminosugars were considered for application in Cystic Fibrosis (CF) [Esposito, A. et al. (2020). Int. J. Mol. Sci. 21, 3353]. Indeed, L-NBDNJ and its congeners have shown anti-inflammatory and antibacterial properties in vitro and in vivo, highlighting the potential of these compounds as therapeutic candidates for the treatment of CF lung disease [De Fenza, M. et al. (2019). Eur. J. Med. Chem. 175, 63; De Gregorio, E. et al. (2020). Antibiotics 9, 362].

### DESCRIPTION OF THE INVENTION

It has now been found that the L-iminosugars having the following structural formulas, identified by the abbreviations L-DNJ, L-NBDNJ, L-AMPDNM and L-MONDNJ and their pharmaceutically acceptable salts exhibit a marked ability to inhibit accumulation of heparan sulfate and therefore are useful tools for the treatment of mucopolysaccharidosis types I, II, III or VII and their subtypes, especially Sanfilippo syndrome and its subtypes A, B, C and D, as well as for the treatment of other conditions characterized by the accumulation of heparan sulfate such as Alzheimer's disease and tumors.

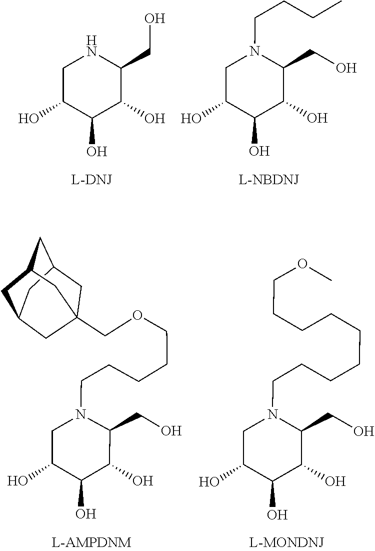

The synthesis of L-NBDNJ (N-butyl-L-deoxynojirimycin, the unnatural enantiomer of Miglustat) is described by D'Alonzo D. et al. (2017). J. Med. Chem. 60, 9462; L-NBDNJ has shown to be a candidate drug for combination therapy of Pompe disease.

The synthesis of the L-iminosugars L-DNJ (unnatural enantiomer of deoxynojirimycin or Duvoglustat) and L-AMPDNM (N-adamantanomethoxypentyl L-DNJ) is described by D'Alonzo D. et al. (2017). J. Med. Chem. 60, 9462 and De Fenza M. et al. (2019). Eur. J. Med. Chem. 175, 63.

The compound L-MONDNJ (N-methoxynonyl L-DNJ) is new and constitutes a further object of the invention.

For the expected therapeutic uses, the iminosugars L-DNJ, L-NBDNJ, L-AMPDNM and L-MONDNJ or their pharmaceutically acceptable salts will be formulated in pharmaceutical compositions suitable for oral or parenteral administration, for example capsules, tablets, solutions and similar, containing suitable excipients.

The dosage will be determined by the specialists based on the patient's conditions, weight, gender and age, as well as by the pharmacokinetic and toxicological characteristics of the compounds. In principle, the dosage may be similar to that of the drugs already in use (Miglustat and Migalastat), for example from 10 to 1000 mg per day, in one or more administrations.

The pharmacological activity observed for the compounds L-DNJ, L-NBDNJ, L-AMPDNM and L-MONDNJ or for their pharmaceutically acceptable salts is very interesting as compared with other structurally similar iminosugars (compounds L-NNDNJ, L-HPDNJ and L-NPDNJ whose formula are reported below), which, evaluated under the same conditions, did not show any activity.

The structural formulas of the compounds according to the invention and of the comparative compounds are reported below.

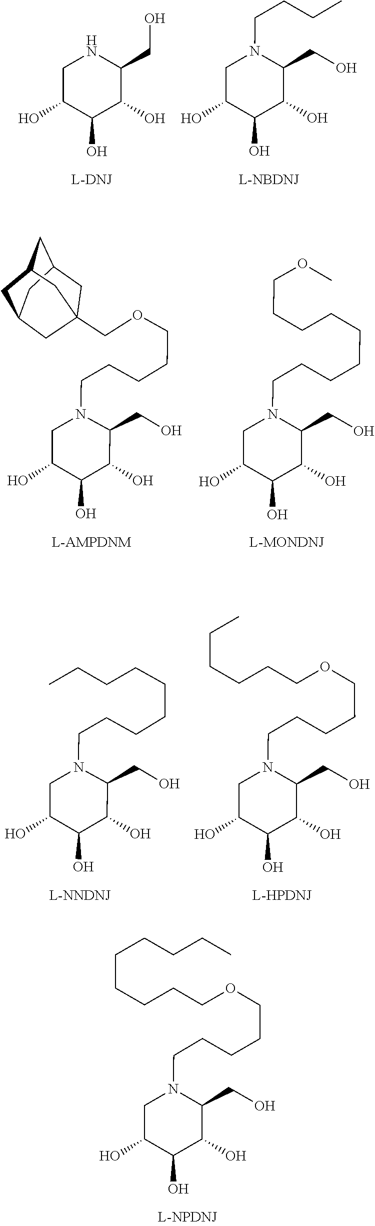

The invention is detailly described in the following experimental part.

### Example 1: Synthesis of L-MONDNJ (N-methoxynonyl L-DNJ) and the Corresponding Hydrochloride Derivative

Step a: Synthesis of 1,9-diiodiononane. Iodine (2.6 g, 10.2 mmol) was added to a stirring suspension of polymer triphenylphosphine (PS-TPP; 100-200 mesh, ˜3 mmol/g triphenylphosphine) (3.4 g, 10.5 mmol) in anhydrous dichloromethane (25 mL) under an argon atmosphere. After 10 minutes, 1,9-nonanediol was added to the suspension (0.41 g, 2.56 mmol) and the reaction was stirred at room temperature for 1 hour. Subsequently the suspension was filtered to remove the polymer-anchored triphenylphosphine oxide by washing with dichloromethane. The filtrate was washed with saturated Na₂S₂O₃, saturated NaCl solution and extracted with dichloromethane.

The organic phase was dried (Na₂SO₄) and evaporated under reduced pressure, giving the desired 1,9-diiodiononane (oil, 0.9 g, 95% yield). 1H NMR (400 MHz, CDCl₃): δ 1.21-1.47 (m, 10H), 1.76-1.88 (m, 4H), 3.19 (t, J=7.0 Hz, 4H). In this reaction, the replacement of the polymeric triphenylphosphine with triphenylphosphine leads to analogous results in terms of reaction time and yield; however, this procedure requires a purification step by precipitation of triphenylphosphine oxide or by chromatography.

Step b: Synthesis of 1-iodo-9-methoxynonane. NaH (60% dispersion in mineral oil, 0.10 g, 2.55 mmol) was added under magnetic stirring to a solution of methanol (0.12 mL, 2.95 mmol) in dry THF (3.5 mL) at 0° C. and under an argon atmosphere. The reaction mixture was stirred at the same temperature for 1 hour; then, a solution of 1,9-diiodiononane (0.75 g, 1.95 mmol) in THF (3.5 mL) was added. The solution was warmed to room temperature and stirred for 48 hours at the same temperature. Subsequently, dichloromethane was added and the solution washed with aqueous NH₄Cl first and then a saturated NaCl solution. The organic phase was dried with Na₂SO₄ and solvent evaporated under reduced pressure. Chromatography of the crude residue on silica gel (hexane:EtOAc=95:5) gave pure 1-iodo-9-methoxynonane (oil, 0.42 g, yield 75%). 1H NMR (400 MHz, CDCl₃): δ 1.21-1.47 (m, 8H), 1.51-1.63 (m, 4H), 1.76-1.88 (m, 2H), 3.19 (t, J=7.0 Hz, 2H), 3.30 (s, 3H), 3.36 (t, J=6.6 Hz, 2H).

Step c: Synthesis of L-MONDNJ (N-methoxynonyl L-DNJ). To a solution of L-DNJ (0.20 g, 1.22 mmol) in dry DMF (4 mL) under magnetic stirring, K₂CO₃ (0.5 g, 3.6 mmol) was added at room temperature under an argon atmosphere. A solution of 1-iodo-9-methoxynonane (0.42 g, 1.46 mmol) in DMF (4.0 mL) was added dropwise and the reaction mixture was heated to 80° C. and stirred for 16 hours. Solvent was removed under reduced pressure and chromatographed on silica gel (acetone:MeOH=8:2) to give pure L-MONDNJ.

Step d: Preparation of L-MONDNJ·HCl (N-methoxynonyl L-DNJ·HCl). L-MONDNJ·HCl hydrochloride was obtained by addition of 1M HCl (1.22 mmol) followed by evaporation under reduced pressure (0.30 g, yield 75%). 1H NMR (500 MHz, CD₃OD): δ 1.28-1.49 (m, 10H), 1.52-1.63 (m, 2H), 1.67-1.88 (m, 2H), 2.95-3.11 (m, 2H), 3.13-3.27 (m, 1H), 3.31 (s, 3H), 3.40 (t, J=6.5 Hz, 4H), 3.47 (dd, J=4.9, 11.8 Hz, 1H), 3.61 (t, J=11.8 Hz, 1H), 3.65-3.77 (m, 1H), 3.91 (d, J=11.6 Hz, 1H), 4.14 (d, J=11.6 Hz, 1H).

### Example 2: Treatment with L-deoxyminosugars (L-DNJ, L-NBDNJ, L-AMPDNM and L-MONDNJ) as Hydrochloride Salt Reduces Lysosomal Defects in a Cellular Model of Sanfilippo B Disease (MPS IIIB)

To study the effects of the compounds on the lysosomal dysfunctions in a neuronal model of Sanfilippo B disease (MPS IIIB), we recently generated stable clones of the human neuroblastoma cell line SK-NBE silenced for the NAGLU gene causative of MPS IIIB [De Pasquale V, et al. (2021). Biochim Biophys Acta Mol Cell Res. 1868, 119113.]. Mimicking the features of Sanfilippo B disease, silencing of NAGLU causes accumulation of heparan sulfate and accumulation of lysosomes in the cytoplasm of stable SK-NBE clones compared to the control (WT) clone.

Therefore, NAGLU-silenced clone (c15) and control clone (WT) were selected to test the effect of L-deoxyminosugars (L-DNJ, L-NBDNJ, L-NNDNJ, L-HPDNJ, L-NPDNJ, L-AMPDNM and L-MONDNJ) in the hydrochloride form on the lysosomal phenotype of our cell model of Sanfilippo B (MPS IIIB).

Clone 5 was cultured in the presence of 20 μM of each L-deoxyminosugar under normal growth conditions and after 48 hours the lysosomal accumulation was evaluated with immunofluorescence technique by using a specific antibody against Lamp1 (lysosomal marker). Untreated clone 5 shows enlarged positive Lamp1 lysosomal structures within the cytoplasm compared to the WT control clone (Table 1). Treatment with L-DNJ, L-NBDNJ, L-AMPDNM, and L-MONDNJ causes a dramatic reduction in lysosomal enlargement and accumulation in the clone 5 (cl5) model system of Sanfilippo B (MPS IIIB) (Table 1). Furthermore, upon treatment with the active L-deoxyminosugar the lysosomes are no longer concentrated in the perinuclear region of the cells, as occurs in several lysosomal diseases, but they are physiologically distributed throughout the cytoplasm. Interestingly, the L-deoxyminosugars L-NNDNJ, L-HPDNJ and L-NPDNJ did not show any activity on the lysosomal phenotype of the Sanfilippo B model clone 5 (c15) (Table 1).

TABLE 1

| | Cells positive for | | Relative Lamp1 | |
|---|---|---|---|---|
| | Lamp1 (%) | | fluorescence intensity | |
| | WT | cl5 | WT | cl5 |
| Mock | 3.5 | 88 | 1.00 | 2.01 |
| L-DNJ•HCl | 2.8 | 25 | 1.00 | 0.95 |
| L-NBDNJ•HCl | 2.6 | 20 | 1.07 | 0.97 |
| L-AMPDNM•HCl | 2.5 | 18 | 1.08 | 0.99 |
| L-MONDNJ•HCl | 2.4 | 21 | 1.05 | 1.00 |
| L-NNDNJ•HCl | 4.1 | 85 | 1.04 | 2.70 |
| L-HPDNJ•HCl | 4.8 | 87 | 1.00 | 2.10 |
| L-NPDNJ•HCl | 3.7 | 84 | 1.10 | 2.65 |

The physiological distribution of lysosomes within the cytoplasm in L-DNJ, L-NBDNJ, L-AMPDNM and L-MONDNJ treated cells was more evident comparing these results with those obtained with the immunofluorescence for Lamp1 protein in the WT control normal clone.

Treatment with any of the seven L-iminosugars did not cause any changes in the size and lysosomal distribution of the non-diseased WT model cells.

Overall, these results show for the first time that treatment with the selected L-iminosugars (L-DNJ, L-NBDNJ, L-AMPDNM and L-MONDNJ) is able to reduce the lysosomal defects in a cellular model of Sanfilippo B disease (MPS IIIB).

### Example 3: Treatment with L-deoxyminosugars (L-DNJ, L-NBDNJ, L-AMPDNM and L-MONDNJ) as Hydrochloride Salt Reduces HS Accumulation in a Cellular Model of Sanfilippo B Disease (MPS IIIB)

Clone 5 was grown in the presence of 20 μM of each L-iminosugar under normal growth conditions and after 48 hours the accumulation of heparan sulfate (HS) was evaluated by immunofluorescence staining for HS. Untreated clone 5 showed an accumulation of HS on the cell membrane compared to the WT control clone (Table 2). In presence of compounds L-DNJ, L-NBDNJ, L-AMPDNM and L-MONDNJ, however, a dramatic reduction of HS staining was observed in the Sanfilippo B model system (MPS IIIB) c15 (Table 2). Also in this case, L-iminosugars L-NNDNJ, L-HPDNJ and L-NPDNJ did not show any activity on the reduction of HS accumulation in the Sanfilippo B model tested (Table 2). These results agree with the results obtained with Lamp1 staining of lysosomes.

TABLE 2

| | | | Relative HS fluorescence | |
|---|---|---|---|---|
| | Cells positive for HS (%) | | intensity | |
| | WT | cl5 | WT | cl5 |
| Mock | 3.2 | 94.9 | 1.0 | 3.5 |
| L-DNJ•HCl | 2.6 | 5.1 | 0.94 | 1.20 |
| L-NBDNJ•HCl | 2.5 | 7.4 | 0.96 | 1.13 |
| L-AMPDNM•HCl | 2.3 | 5.1 | 0.94 | 1.19 |
| L-MONDNJ•HCl | 2.1 | 4.9 | 0.97 | 1.15 |
| L-NNDNJ•HCl | 4.1 | 97.5 | 0.94 | 3.71 |
| L-HPDNJ•HCl | 2.4 | 95.1 | 0.95 | 3.60 |
| L-NPDNJ•HCl | 4.4 | 97.5 | 0.93 | 3.65 |

Furthermore, treatment with any of the seven L-iminosugars caused no change in the amounts of HS in the non-diseased WT model cells.

Overall, these results show for the first time that the treatment with the selected L-iminosugars L-DNJ, L-NBDNJ, L-AMPDNM and L-MONDNJ is able to reduce HS accumulation in a cellular model of the Sanfilippo B disease (MPS IIIB) generated in our laboratory.

### Example 4: Treatment with L-deoxyminosugars (L-DNJ, L-NBDNJ, L-AMPDNM and L-MONDNJ) as Hydrochloride Salt Reduces Lysosomal Defects and HS Accumulation in Fibroblasts of Patients Affected by Sanfilippo A and B (MPS IIIA and IIIB)

To verify whether the selected iminosugars could exert the same effects also on the fibroblasts of patients affected by Sanfilippo disease, human adult dermal fibroblasts HDFa (purchased from Sigma-Aldrich) were used as control, and fibroblasts of patients affected by Sanfilippo A and B (MPS IIIA and IIIB) were used as disease model.

The human cell lines, fibroblasts, from patients with MPS (Sanfilippo disease) used in the examples were obtained from the G. Gaslini Institute of Genoa, “Cell Line and DNA Biobank from Patients Affected by Genetic Diseases”-Telethon Genetic Biobank Network—Telethon research service. These cells are classified with identification codes and by type of disease without allowing patient identification. Cells were collected from the patients, at the Gaslini Institute, with informed consent to the collection extended to conservation and its possible use, for diagnosis and/or research purposes according to current legislation as required by the guidelines followed by the Telethon biobanks. The Network operates abiding by the Italian Privacy and Data Protection Laws in force, including: Italian Data Protection Authority, Personal Data Protection Code, Legislative Decree no. 196, 30 Jun. 2003, published in Official Gazette No. 174 of the Italian Republic, 29 Jul. 2003; Italian Data Protection Authority, General Authorization for the processing of genetic data, 24 Jun. 2011, published in Official Gazette No. 159 of Italian Republic, 11 Jul. 2011.

Cells were cultured in the presence of the selected L-iminosugars at a dosage of 20 μM and after 48 hours were treated for HS and Lamp1 immunofluorescence. Treatment with L-iminosugars had no effect on control HDFa (Table 3). On the other hand, the same L-iminosugars L-DNJ, L-NBDNJ, L-AMPDNM and L-MONDNJ caused a strong reduction of HS and lysosome accumulation in fibroblasts of patients affected by Sanfilippo A and B (MPS IIIA and MPS IIIB) (Table 3).

TABLE 3

| | Relative fluorescence intensity compared to HDFa | | | | | |
|---|---|---|---|---|---|---|
| | HDFa | | MPS-IIIA | | MPS-IIIB | |
| | Lamp1 | HS | Lamp1 | HS | Lamp1 | HS |
| Mock | 1.0 | 1.0 | 3.0 | 5.3 | 3.0 | 6.0 |
| L-DNJ•HCl | 1.0 | 1.0 | 1.8 | 1.0 | 1.0 | 1.0 |
| L-NBDNJ•HCl | 1.0 | 1.0 | 1.9 | 1.7 | 1.0 | 1.5 |
| L-AMPDNM•HCl | 1.0 | 1.0 | 2.2 | 1.5 | 1.0 | 1.0 |
| L-MONDNJ•HCl | 1.0 | 1.0 | 2.2 | 1.8 | 1.3 | 1.7 |
| L-NNDNJ•HCl | 1.0 | 1.0 | 3.0 | 4.9 | 3.1 | 5.8 |
| L-HPDNJ•HCl | 1.0 | 1.0 | 3.1 | 5.4 | 3.0 | 5.7 |
| L-NPDNJ•HCl | 1.0 | 1.0 | 2.8 | 5.2 | 3.3 | 5.9 |

Also in this case, L-iminosugars L-NNDNJ, L-HPDNJ and L-NPDNJ did not show any activity on the reduction of HS and Lamp1 accumulation in the Sanfilippo A and B patient fibroblasts (Table 3).

Overall, these results show for the first time that treatment with the selected L-iminosugars L-DNJ, L-NBDNJ, L-AMPDNM and L-MONDNJ is able to prevent the accumulation of HS and lysosomes in the fibroblasts of patients affected by Sanfilippo A and B disease.

### Example 5: Treatment with D-deoxyminosugars (D-DNJ or DNJ, D-NBDNJ or NBDNJ or D-AMPDNM or AMPDNM, or D-AMPDNM o AMPDNM, e D-MONDNJ o MONDNJ) as Hydrochloride Salts has No Effect on Lysosomal Defects and HS Accumulation in Fibroblasts of Patients Affected by Sanfilippo A and B (MPS IIIA and IIIB)

To demonstrate that the iminosugars currently available for the treatment of other lysosomal diseases (D-DNJ also known as Duvoglustat and D-NBDNJ known as Miglustat) and that stereoisomers D-AMPDNM and D-MONDNJ do not have the same efficacy as the compounds object of this invention, human adult dermal fibroblasts HDFa were used as controls (purchased from SIGMA), and fibroblasts from patients with Sanfilippo A and B (MPS IIIA and IIIB) were used as disease models. Cells were grown in the presence of the iminosugars D-DNJ, D-NBDNJ, D-AMPDNM and D-MONDNJ at the dosage of 20 μM and after 48 hours were treated for HS and Lamp1 immunofluorescence. Treatment with D-iminosugars had no effect on HDFa and fibroblasts of patients affected by Sanfilippo A and B (MPS IIIA and MPS IIIB) both on HS and lysosome accumulation (Table 4).

TABLE 4

| | Cells positive for Lamp1 (%) | | | Cells positive for HS (%) | | |
|---|---|---|---|---|---|---|
| | HDFa | MPS-IIIA | MPS-IIIB | HDFa | MPS-IIIA | MPS-IIIB |
| Mock | 5.5 | 94.8 | 95.0 | 4.5 | 97.5 | 96.8 |
| D-DNJ•HCl | 5.0 | 95.0 | 92.5 | 2.4 | 97.3 | 94.9 |
| D-NBDNJ•HCl | 5.0 | 94.7 | 93.8 | 4.8 | 99.7 | 95.2 |
| D-AMPDNM•HCl | 5.1 | 96.8 | 94.6 | 4.9 | 98.7 | 96.4 |
| D-MONDNJ•HCl | 5.3 | 95.2 | 93.8 | 5.0 | 99.2 | 95.8 |

Overall, these results show the efficacy of the selected L-iminosugars L-DNJ, L-NBDNJ, L-AMPDNM and L-MONDNJ in preventing HS and lysosome accumulation in cellular model of Mucopolysaccharidoses such as fibroblasts from patients affected by Sanfilippo A and B.

### Example 6: Treatment with L-deoxyminosugars (L-DNJ, L-NBDNJ, L-AMPDNM and L-MONDNJ) in the Form of Hydrochlorides Salts Triggers the Reduction of the Amounts of HS in the HeLa Tumor Epithelial Cell Line and Reduction of their Growth

To test whether treatment with the selected L-deoxyminosugars (L-DNJ, L-NBDNJ, L-AMPDNM and L-MONDNJ) could interfere with HS synthesis we selected a cell line not mutated for NAGLU and highly decorated by heparan sulfate on the cell membrane. For this purpose, HeLa tumor epithelial cells (purchased from ATCC) were cultured for 48 hours in the presence of the selected L-iminosugars and the amount of HS was evaluated by immunostaining. Treatment with the same L-iminosugars L-DNJ, L-NBDNJ, L-AMPDNM and L-MONDNJ caused a strong reduction of HS accumulation on the HeLa cell membrane (Table 5).

TABLE 5

| | Relative HS fluorescence |
|---|---|
| | intensity |
| mock | 1.00 |
| L-DNJ•HCl | 0.24 |
| L-NBDNJ•HCl | 0.26 |
| L-AMPDNM•HCl | 0.12 |
| L-MONDNJ•HCl | 0.11 |
| L-NNDNJ•HCl | 0.88 |
| L-HPDNJ•HCl | 0.95 |
| L-NPDNJ•HCl | 1.19 |

Also in this case, L-iminosugars L-NNDNJ, L-HPDNJ and L-NPDNJ did not show any activity on the reduction of HS accumulation in the HeLa tumor epithelial cells (Table 5).

Furthermore, treatment with the L-deoxyminosugars L-DNJ, L-NBDNJ, L-AMPDNM and L-MONDNJ caused a decrease of the number of HeLa cells after 48 hours as shown by the cell proliferation assay reported in Table 6.

TABLE 6

| | Cell number |
|---|---|
| mock | 5.2 · 10⁵ |
| L-DNJ•HCl | 2.9 · 10⁵ |
| L-NBDNJ•HCl | 3.6 · 10⁵ |
| L-AMPDNM•HCl | 3.7 · 10⁵ |
| L-MONDNJ•HCl | 3.1 · 10⁵ |
| L-NNDNJ•HCl | 4.2 · 10⁵ |
| L-HPDNJ•HCl | 4.3 · 10⁵ |
| L-NPDNJ•HCl | 4.7 · 10⁵ |
| D-DNJ•HCl | 4.4 · 10⁵ |
| D-NBDNJ•HCl | 4.2 · 10⁵ |
| D-AMPDNM•HCl | 5.1 · 10⁵ |
| D-MONDNJ•HCl | 5.0 · 10⁵ |

This result demonstrates that treatment with L-DNJ L-iminosugars, L-NBDNJ, L-AMPDNM and L-MONDNJ causes a reduction of HS in HeLa cancer cells followed by an inhibition of tumor cell growth, since HS is essential to sustain the proliferation of cancer epithelial cells.

The data reported in Table 6 also demonstrate that the iminosugars belonging to D series, D-DNJ or Duvoglustat, D-NBDNJ or Miglustat, D-AMPDNM and D-MONDNJ do not have the same efficacy compared to the compounds object of this invention on the reduction of cancer cell proliferation.

These data demonstrate that the L-iminosugars act by interfering with HS synthesis with a completely new mechanism compared to the mechanism of action of other commercially available iminosugars. Furthermore, these results show that the invention can be applied not only for mucopolysaccharidosis where there is an accumulation of HS, but also for cancer diseases where HS is essential to support the growth of tumor cells and their metastatic mechanisms.

### Example 7: Treatment with L-deoxyminosugars (L-DNJ, L-NBDNJ, L-AMPDNM and L-MONDNJ) in the Form of Hydrochloride Salt Triggers Reduction of the Amounts of Amyloid Beta Fiber in a Cellular Model of Sanfilippo B Disease (MPS IIIB)

In order to demonstrate the therapeutic applications of the selected iminosugars L-DNJ, L-NBDNJ, L-AMPDNM and L-MONDNJ for neurodegenerative diseases we used the neuronal model of Sanfilippo B (c15), recently generated in our laboratory (example 2).

The clone (c15) stably silenced for NAGLU, the causative gene of MPS IIIB, is able to mimic the features of neurodegenerative diseases as it accumulates beta-amyloid fibers in the cytoplasm. In fact, Sanfilippo Syndrome is also defined as “childhood Alzheimer”.

The diseased clone (c15) was grown in the presence of L-DNJ, L-NBDNJ, L-AMPDNM and L-MONDNJ and the corresponding enantiomers D-DNJ, D-NBDNJ, D-AMPDNM and D-MONDNJ at a dosage of 20 μM and after 48 hours were processed by immunofluorescence against the beta-amyloid peptide 1-42. The data reported in Table 7 show that treatment with the L-deoxyminosugars lead to a total reduction in the accumulation of amyloid fibers compared to the untreated cells (mock). On contrary, treatment with the corresponding D-enantiomers has no effect on the reduction of the accumulation of amyloid fibers compared to the untreated clone (mock) (Table 7).

TABLE 7

| | Relative amyloid fluorescence |
|---|---|
| | intensity |
| mock | 1.00 |
| L-DNJ•HCl | 0.01 |
| L-NBDNJ•HCl | 0.01 |
| L-AMPDNM•HCl | 0.02 |
| L-MONDNJ•HCl | 0.01 |
| D-DNJ•HCl | 0.99 |
| D-NBDNJ•HCl | 0.98 |
| D-AMPDNM•HCl | 1.02 |
| D-MONDNJ•HCl | 1.05 |

These results show for the first time that L-iminosugars object of the claims, L-DNJ, L-NBDNJ, L-AMPDNM and L-MONDNJ, are effective for the reduction of accumulation of amyloid plaques and for the treatment of neurodegenerative processes such as Alzheimer's. The data reported in Table 7 also demonstrate that D-iminosugars D-DNJ, D-NBDNJ, D-AMPDNM and D-MONDNJ, do not have the same efficacy on the reduction of neurodegeneration markers, differently from the compounds object of this invention.

1. Method of treating or preventing diseases characterized by heparan sulfate accumulation in a subject in need thereof with iminosugars of formula: 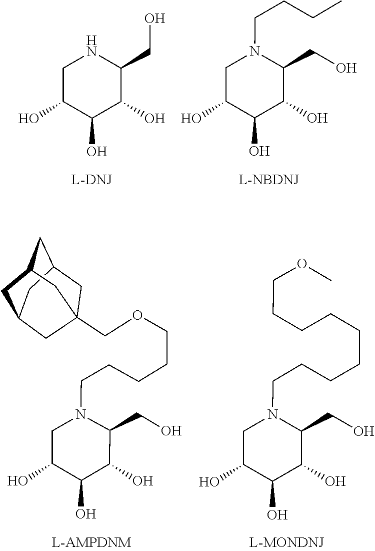
or pharmaceutically acceptable salts thereof, wherein said diseases are selected among mupolysaccharidosis, Alzheimer's and cancers, said method comprising:
administering to said subject a pharmaceutical acceptable amount of said iminosugars.

2. The method to claim 1 wherein the heparan sulfate storage disease is mucopolysaccharidosis type I, II, III or VII.

3. The method to claim 2 wherein the disease is Sanfilippo syndrome and its subtypes (A, B, C, D).

4. The method to claim 2 wherein the disease is Alzheimer's disease.

5. The method according to claim 2 wherein the disease is a neoplasm.

6. Compound of formula: 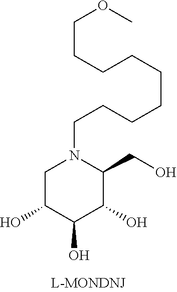 [SMILES: COCCCCCCCCCN1C[C@@H](O)[C@H](O)[C@@H](O)[C@@H]1CO]
and its pharmaceutically acceptable salts.
